(19) United States
(12) Patent Application Publication
(10) Pub. No.: US 2003/0133896 A1
(43) Pub. Date: Jul. 17, 2003

(54) Cosmetic and pharmaceutical oil-in-water emulsions

(75) Inventors: Thomas Dietz, Essen (DE); Peter Hameyer, Essen (DE); Ute Schick, Koln (DE)

Correspondence Address: Scully, Scott, Murphy & Presser, 400 Garden City Plaza, Garden City, NY, 11530, US

(73) Assignee: Goldschmidt AG, Essen (DE)

(21) Appl. No.: 10/336,350

(22) Filed: Jan. 3, 2003

(30) Foreign Application Priority Data
Jan. 4, 2002 (DE) 102 00 209.6

(51) Int. Cl. A61K 7/06; A61K 7/11

(52) U.S. Cl. 424/70.14

(57) ABSTRACT

Cosmetic or pharmaceutical oil-in-water emulsions which include one or more hydrophobically modified copolymeric polyglutamic acid derivatives; and auxiliaries and additives are provided. The copolymeric polyglutamic acid derivatives used are compounds which are prepared by simultaneous or stepwise reaction of glutamic acid and at least one further α-amino acid and/or derivatives thereof and amines, in the absence of solvents and catalysts.

### FIELD OF THE INVENTION

The present invention relates to oil-in-water emulsions, and more particularly to the use of hydrophobically modified copolymeric polyglutamic acid derivatives of defined structure for the preparation of cosmetic or pharmaceutical oil-in-water emulsions. The present invention also relates to oil-in-water emulsions which comprise such hydrophobically modified coploymeric polyglutamic acid derivatives.

### BACKGROUND OF THE INVENTION

Hydrophobically modified polyamino acid derivatives have been known for a long time, as has their use for the preparation of cosmetic or pharmaceutical emulsions. Thus, for example, DE-A-22 53 190 discloses the use of polyaspartamides as surfactants or interface-active compounds, in particular laundry detergents and cosmetics which comprise these polyaspartamides. This prior art reference also provides a skin cream as an example which comprises 25% of a polyaspartamide.

DE-A-195 24 097 discloses cosmetic compositions which, apart from alkyl and/or alkenyl oligoglycosides and/or fatty acid-N-alkylpolyhydroxyalkylamides, also comprise hydrophobicized oligopeptides. One group of hydrophobicized oligopeptides represent reaction products of polyaspartic acids with amines having 1 to 22 carbon atoms. The addition of the hydrophobicized oligopeptides to selected sugar surfactants leads to the establishment of an advantageously high viscosity, a synergistic improvement in the base foam and the foam stability. In addition, these prior art compositions impart a pleasant feel on the skin.

DE-A-195 45 678 describes copolymeric polyamino acid esters and use thereof inter alia as emulsifiers in cosmetic preparations. The examples given are O/W (i.e., oil-in-water) or W/O (i.e., water-in-oil) emulsions of simple construction, consisting of water, emulsifier and oil, which were stable for at least 3 weeks.

DE-A-197 20 771 provides a process for the preparation of polyaspartamides and use thereof for the preparation of cosmetic and/or pharmaceutical preparations. These preparations may, for example, be hair shampoos, hair lotions, foam baths, creams, lotions or ointments.

EP-A-958 811 discloses cosmetic O/W emulsions comprising one or more hydrophobically modified polyaspartic acid derivatives, one or more bodying agents, and optionally additional coemulsifiers, and customary auxiliaries and additives. These prior art O/W emulsions can be used as skincare compositions, daycream, nightcream, care cream, nutrient cream, body lotion, pharmaceutical ointment and lotion, aftershave lotion and sunscreen.

Emulsifiers are required for the preparation of emulsions. Emulsifiers are surfactants with at least one hydrophilic and at least one hydrophobic or lipophilic moiety. The hydrophobic moiety or lipophilic moiety is formed by saturated or unsaturated, linear or branched alkyl radicals having primarily 12 to 22 carbon atoms, or polypropylene glycol or polydimethylsiloxane. The emulsifiers can be divided, depending on the chemical structure of the hydrophilic moiety, into nonionogenic, anion-active, cation-active or amphoteric emulsifiers. Examples of anion-active hydrophilic groups are neutralized carboxyl, sulfate or phosphate groups. Examples of anion-active O/W emulsifiers are self-emulsifying glyceryl stearate, which is obtained by partial saponification of glycerol monodistearate with potassium hydroxide, the potassium stearate (soap) formed in this process acts as an emulsifying-active component. Further examples of anion-active O/W emulsifiers include amine soaps, such as triethanolammonium stearate, sodium cetearyl sulfate, potassium cetyl phosphate, sodium glyceryl stearate citrate and sodium stearoyl lactylate. A particular advantage of anion-active emulsifiers is their extraordinarily high emulsifiability. A decisive disadvantage, however, is the limitation of the pH range of emulsions prepared therewith. With self-emulsifying glyceryl stearate, for example, it is only possible to prepare emulsions with a pH of >6.8, since the emulsifying-active component is ineffective at a lower pH. However, cosmetic oil-in-water emulsions are desired whose pH corresponds to the natural pH of the skin, i.e., about 5.5.

In view of the drawbacks mentioned with the prior art, there is still a need to provide new and improved anion-active emulsifiers that can be used in preparing cosmetic or pharmaceutical oil-in-water emulsions having a skin friendly pH of 5.5.

### SUMMARY OF THE INVENTION

An object of the present invention is to provide anion-active emulsifiers with which cosmetic or pharmaceutical oil-in-water emulsions having a skin-friendly pH of 5.5 can be prepared, which are also characterized by having a good high-temperature and low-temperature stability, a brilliant appearance, and a pleasant feel on the skin. In addition, the emulsifiers of the present invention include renewable raw materials, not any oxidation-sensitive radicals, such as, for example, polyethylene glycol radicals, and the inventive emulsfiers are biodegradable.

Surprisingly, it has now been found that, using hydrophobically modified copolymeric polyglutamic acid derivatives of a certain composition, it is possible to prepare oil-in-water emulsions with a very fine degree of dispersion, synonymous with a brilliant appearance, with very good low-temperature and high-temperature stability whose pH can be adjusted to that of the natural pH of the skin. The inventive emulsifiers are based on vegetable raw materials. Moreover, the emulsifiers of the present invention are not oxidation-sensitive and are biodegradable. It has also been found that the inventive emulsifiers are highly effective oil-in-water emulsifiers even in a very low concentration of <1%, if the emulsfiers are preferably combined/with wax-like bodying agents (“polar waxes” or “hydrophilic waxes”), such as glyceryl monodistearate, stearyl alcohol, cetyl alcohol or stearic acid.

The present invention therefore provides cosmetic or pharmaceutical oil-in-water emulsions which comprise one or more hydrophobically modified copolymeric polyglutamic acid derivatives; and auxiliaries and additives, in which the copolymeric polyglutamic acid derivatives used are compounds that are prepared by simultaneous or stepwise reaction of glutamic acid and at least one further α-amino acid and/or derivatives thereof and amines in the absence of solvents and catalysts by processes known per se.

The present invention further provides cosmetic or pharmaceutical oil-in-water emulsions which comprise

(a) one or more hydrophobically modified copolymeric polyglutamic acid derivatives prepared in accordance with the present invention;

(b) one or more polar waxes selected from the group consisting of fatty alcohols having 12 to 22 carbon atoms, fatty acids having 12 to 22 carbon atoms and/or glycerol or polyglycerol partial esters of fatty acids having 12 to 22 carbon atoms; and

(c) one or more cosmetic oils.

The present invention further provides cosmetic or pharmaceutical oil-in-water emulsions, wherein the proportion of component (a) is between 0.1 and 2.0%, the proportion of component (b) is 0.5 to 8.0%, and the proportion of component (c) is 1.0 to 60% of the overall emulsion.

### DETAILED DESCRIPTION OF THE INVENTION

As stated above, the present invention provides emulsifers which are based on one or more hydrophobically modified copolymeric polyglutamic acids derivatives.

In accordance with the present invention, 30 to 90% by weight of the units present in the copolymers are structural units of general formula (I) and 70 to 30% by weight of the units in the copolymers are of general formula (II)

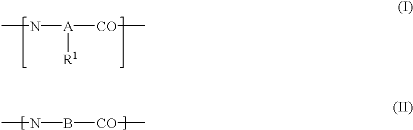

in which

A is a trifunctional radical with three carbon atoms and has one of the following structures:

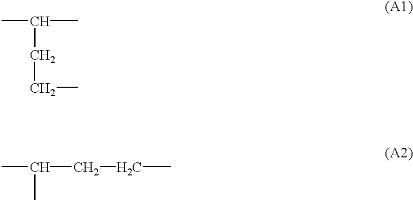

and in which

R¹ can have the meaning of R², R³ and R⁴,

where

R² represents identical or different amide radicals —C(O)NH—R⁹ where

R⁹ represents straight-chain or branched, saturated and unsaturated alkyl radicals having 1 to 24, preferably 6 to 24, carbon atoms or radicals of the structure —C(O)NH—X—R⁹, where

X is an oligo- or polyoxyalkylene chain having 1 to 100 oxyalkylene units, preferably ethylene oxide units,

R³ represents identical or different amide radicals —C(O)NH—(CH₂)n-N(R¹⁰)(R¹¹) where

n is 2 to 10, preferably 2 to 4, and

R¹⁰, R¹¹, independently of one another, represent straight-chain or branched, saturated or unsaturated alkyl radicals having 1 to 24 carbon atoms and/or hydroxyalkyl radicals,

R⁴ has the meaning COO⁻X⁺, where

X⁺ represents one or more radicals from the group of alkali metals, alkaline earth metals, hydrogen or ammonium, [NR⁵R⁶R⁷R⁸]⁺, in which

R⁵ to R⁸, independently of one another, represent hydrogen, alkyl or hydroxyalkyl or [NH₃—X—R⁹]⁺, and/or [NH₃—R⁹]⁺, and/or [NH₃—(CH₂)ₙ—N(R¹⁰)(R¹¹)]⁺,

and at least in each case one radical R¹ has the meaning of R², R³ and/or R⁴, and the units [—NH—B—CO—] are building blocks from the group of proteinogenic and/or nonproteinogenic amino acids (H₂N—B—COOH), in which B is the radical of the corresponding amino acid.

Suitable amino acid building blocks [—NH—B—CO] from the group of proteinogenic amino acids H₂N—B—COOH are, for example, glycine, alanine, leucine, isoleucine, phenylalanine, tyrosine, serine, cysteine, methionine, glutamic acid, glutamine, aspartic acid, asparagine, lysine, hydroxylysine, arginine, tryptophan, histidine, valine, threonine, proline, hydroxyproline and derivatives thereof; nonproteinogenic amino acids may, for example, be β-alanine, ω-amino-1-alkanoic acids and the like.

Suitable amines which can be co-used according to the present invention to prepare the copolymers are compounds which contain at least one amino group which can react with carboxyl groups, such as, for example:

NH₂—R⁹ and/or H₂N—X—R⁹ and/or H₂N—(CH₂)ₙ—N(R¹⁰)(R¹¹) in which R⁹, R¹⁰, R¹¹, X and n have the meanings given above.

Preference is given to using those compounds which are liquid at the reaction conditions given and do not distill off from the reaction mixture. Examples of such compounds include the higher amines having 6 or more carbon atoms in the alkyl chain, such as the homologous series of fatty amines H₂N—R⁹.

These fatty amines are prepared by known processes, such as, for example, by reacting fatty acids with NH₃ in the presence of catalysts to give the nitrile and subsequent hydrogenation to give the primary or secondary amine.

The fatty acids co-used, individually or in mixtures, are acids, such as, caprylic acid, capric acid, 2-ethylhexanoic acid, lauric acid, myristic acid, palmitic acid, palmitoleic acid, isostearic acid, stearic acid, hydroxystearic acid (ricinoleic acid), dihydroxystearic acid, oleic acid, linoleic acid, petroselic acid, elaidic acid, arachidic acid, behenic acid and erucic acid, gadoleic acid, and the technical-grade mixtures which are produced during the pressurized cleavage of natural fats and oils, such as, oleic acid, linoleic acid, linolenic acid, and, in particular, rapeseed oil fatty acid, soybean oil fatty acid, sunflower oil fatty acid, tall oil fatty acid. In principle, all fatty acids with a similar chain distribution are suitable for use in the present invention.

The content of unsaturated proportions in the fatty acids or fatty acid esters is, where necessary, set to a desired iodine number through known catalytic hydrogenation processes, or by mixing completely hydrogenated fatty components with nonhydrogenated fatty components. The iodine number, being a measure of the average degree of saturation of a fatty acid, is the amount of iodine which is taken up by 100 g of the compound to saturate the double bonds.

Preference is given to using partially hydrogenated C₈-C₁₈-coconut or palm fatty acids, rapeseed oil fatty acids, sunflower oil fatty acids, soybean oil fatty acids and tall oil fatty acids with iodine numbers in the range from about 80 to 150 and, in particular, technical-grade C₈-C₁₈-coconut fatty acids, where, in some instances, a choice of cis/trans isomers, such as elaidic acid-rich C₁₆-C₁₈-fatty acid cuts, may be advantageous. The above-mentioned fatty acids are standard commercial products and are supplied by various companies under their respective trade names.

Further amines which can be co-used in the present invention are those of the general formula H₂N—(CH₂)ₙ—N(R¹⁰)(R¹¹), which, in addition to a primary amino group, also contain at least one secondary or preferably tertiary amino group, such as, in particular, dimethylaminopropylamine (DMAPA).

Further amines which can be co-used in the present invention are those of the general formula H₂N—X—R⁹, such as, in particular, the compounds sold by Huntsman under the trade name Jeffamine®.

Further amines which can be co-used are compounds which contain imidazoline rings, such as, for example, those from the above-listed fatty acids and diethylenetriamine which are prepared by known methods.

In the copolymers co-used according to the present invention, the glutamic acid/coamino acids ratio is: 90/10 to 30/70% by weight, preferably 90/10 to 50/50% by weight, and the amino acids/amine ratio is: 90/10 to 30/70% by weight, preferably 40/60 to 60/40.

The copolymers co-used according to the present invention are prepared by simultaneous or stepwise reaction of glutamic acid and at least one further α-amino acid and/or derivatives and amines thereof in the absence of solvents and catalysts, under condensation conditions.

One process for the preparation of the copolymeric polypeptides consists, in a first stage, in reacting glutamic acid and/or derivatives thereof in the absence of solvents and catalysts, under condensation conditions, with amines, where necessary at subatmospheric pressure and with removal of the condensate from the reaction mixture and, in a second stage, adding one or more further amino acids and derivatives thereof and/or one or more identical and/or further amines simultaneously, or in any desired order, and reacting them in the absence of solvents and catalysts, under condensation conditions, where necessary at subatmospheric pressure and with removal of the condensate from the reaction mixture.

A further process for the preparation of the copolymeric polypeptides consists, in a first stage, in reacting glutamic acid and/or derivatives thereof with one or more further α-amino acids and/or derivatives thereof, in the absence of solvents and catalysts, under condensation conditions, where necessary at subatmospheric pressure and with removal of the condensate from the reaction mixture and, in a second stage, reacting one or more amines simultaneously, or in any desired order, in the absence of solvents and catalysts, under condensation conditions, where necessary at subatmospheric pressure and with removal of the condensate from the reaction mixture, with the reaction product of the first stage.

A further process for the preparation of the copolymeric polypeptides consists, in a first stage, in reacting glutamic acid and/or derivatives thereof with one or more further α-amino acids and/or derivatives thereof, with one or more amines simultaneously, or in any desired order, in the absence of solvents and catalysts, under condensation conditions, where necessary at subatmospheric pressure and with removal of the condensate from the reaction mixture and, in a second stage, reacting glutamic acid and/or one or more further coamino acids and/or derivatives thereof and/or one or more identical and/or further amines simultaneously, or in any desired order, in the absence of solvents and catalysts, under condensation conditions, where necessary at subatmospheric pressure and with removal of the condensate from the reaction mixture, with the reaction product of the first stage.

A further process for the preparation of the copolymeric polypeptides consists, in a first stage, in mixing at least one amino acid and/or derivatives thereof with, where appropriate, amines in a ratio such that the mixtures are liquid at the condensation temperature, and heating them in the absence of solvents and catalysts to at least the melting temperature and, in a second stage, optionally metering in further coamino acids and/or amines simultaneously, or in any desired order, and reacting them under condensation conditions, where necessary at subatmospheric pressure and with removal of the condensate from the reaction mixture, with the mixture or the reaction product of the first stage.

In addition to the hydrophobically modified polyglutamic acid derivatives used according to the present invention, it is also possible to use other emulsifiers customary in cosmetics. Suitable further emulsifiers are, for example, nonionogenic surfactants selected from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group

C₁₂-C₁₈-fatty acid mono- and diesters of addition products of from 1 to 30 mol of ethylene oxide onto glycerol

glycerol mono- and diesters and sorbitan mono- and diesters of saturated and unsaturated fatty acids having 6 to 22 carbon atoms and ethylene oxide addition products thereof

alkyl mono- and oligoglycosides having 8 to 22 carbon atoms in the alkyl radical and ethoxylated analogs thereof

addition products of from 15 to 60 mol of ethylene oxide onto castor oil and/or hydrogenated castor oil

polyol and, in particular, polyglycerol esters, such as, for example, polyglycerol polyricinoleate, polyglycerol-12-hydroxystearate or polyglycerol dimerate. Likewise suitable are mixtures of compounds from two or more of these classes of substance

addition products of from 2 to 15 mol of ethylene oxide onto castor oil and/or hydrogenated castor oil

partial esters based on linear, branched, unsaturated or saturated C₆-C₂₂-fatty acids, cinoleic acid, and 12-hydroxystearic acid and glycerol, polyglycerol, pentaerythritol, dipentaerythritol, sugar alcohols (for example, sorbitol), alkyl glucosides (for example, methyl glucoside, butyl glucoside, lauryl glucoside), and polyglucosides (for example, cellulose)

mono-, di- and trialkyl phosphates, and mono-, di- and/or tri-PEG-alkyl phosphates and salts thereof

wool wax alcohols

polysiloxane-polyalkyl-polyether copolymers or corresponding derivatives

mixed esters of pentaerythritol, fatty acids, citric acid and fatty alcohol as in DE-B-11 65 574 and/or mixed esters of fatty acids having 6 to 22 carbon atoms, methyl glucose and polyols, preferably glycerol or polyglycerol, and

polyalkylene glycols

betaines

ester quats

sodium, potassium or ammonium salts of long-chain alkyl and alkyl ether sulfonic acids

The addition products of ethylene oxide and/or of propylene oxide onto fatty alcohols, fatty acids, alkylphenols, glycerol mono- and diesters, and also sorbitan mono- and diesters of fatty acids or onto castor oil represent known commercially available products. These are homolog mixtures whose average degree of alkoxylation corresponds to the ratio of the quantitative amounts of ethylene oxide and/or propylene oxide and substrate with which the addition reaction is carried out.

Furthermore, zwitterionic surfactants can be used as emulsifiers. Zwitterionic surfactant is the term used to refer to those surface-active compounds which carry at least one quaternary ammonium group and at least one carboxylate and one sulfonate group in the molecule. Particularly suitable zwitterionic surfactants are, the so-called betaines, such as, the N-alkyl-N,N-dimethylammonium glycinates, for example, cocoalkyldimethyl-ammonium glycinate, N-acylaminopropyl-N,N-dimethylammonium glycinates, for example, cocoacylaminopropyldimethylammonium glycinate, and 2-alkyl-3-carboxylmethyl-3-hydroxyethylimidazolines having, in each case, 8 to 18 carbon atoms in the alkyl or acyl group, and cocoacylaminoethyl hydroxyethylcarboxymethylglycinate. Particular preference is given to the fatty acid amide derivative known under the CTFA name Cocamidopropyl Betaine.

Likewise suitable emulsifiers are ampholytic surfactants. Ampholytic surfactants are understood as meaning those surface-active compounds which, apart from a C₈-C₁₈-alkyl or -acyl group in the molecule, contain at least one free amino group and at least one COOH or SO₃H group and are capable of forming internal salts. Examples of suitable ampholytic surfactants include: N-alkylglycines, N-alkylpropionic acids, N-alkylamino-butyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having, in each case, about 8 to 18 carbon atoms in the alkyl group. Particularly preferred ampholytic surfactants are N-cocoalkylaminopropionate, cocoacylaminoethylaminopropionate and C₁₂-C₁₈-acylsarcosine.

In addition to the ampholytic emulsifiers, quaternary emulsifiers are also suitable. Quaternary emulsifiers of the ester quat type are particularly preferred. Preferably the quaternary emulsifier is a methyl-quaternized difatty acid triethanolamine ester salt.

Suitable bodying agents (hydrophilic or polar waxes) which can be employed in the present invention are primarily fatty alcohols or hydroxy fatty alcohols having 12 to 22 and preferably 16 to 18 carbon atoms, and also partial glycerides, fatty acids or hydroxy fatty acids. Suitable thickeners (hydrocolloids) which can be employed in the present invention are, for example, polysaccharides, in particular, xanthan gum, guar guar, agar agar, alginates and Tyloses, carboxymethylcellulose and hydroxyethylcellulose, and also higher molecular weight polyethylene glycol mono- and diesters of fatty acids, polyacrylates (for example Carbopols from Goodrich, TEGO carbomers from Goldschmidt or Synthalens from Sigma), polyacrylamides, polyvinyl alcohol and polyvinylpyrrolidone, surfactants such as, for example, ethoxylated fatty acid glycerides, esters of fatty acids with polyols such as, for example, pentaerythritol or trimethylolpropane, fatty alcohol ethoxylates having a narrowed homolog distribution, or alkyl oligoglucosides.

Suitable as the oil phase are, for example, those oil components which are known as cosmetic and pharmaceutical oil components and as components of lubricants. These include, in particular, mono- or diesters of carbonic acid (carbonates) and of linear and/or branched mono- and/or dicarboxylic acids having 2 to 44 carbon atoms with linear and/or branched saturated or unsaturated alcohols having 1 to 22 carbon atoms. Also suitable within the meaning of the present invention are the esterification products of aliphatic difunctional alcohols having 2 to 36 carbon atoms with monofunctional aliphatic carboxylic acids having 1 to 22 carbon atoms. Monoesters suitable as oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters obtainable from technical-grade aliphatic alcohol cuts and technical-grade, aliphatic carboxylic acid mixtures, for example, esters of unsaturated fatty alcohols having 12 to 22 carbon atoms and saturated and unsaturated fatty acids having 12 to 22 carbon atoms, as are accessible from animal and vegetable fats. Also suitable, however, are naturally occurring monoester or wax ester mixtures, as are present, for example, in jojoba oil or in sperm oil.

Suitable esters are, for example, carbonic diesters (dialkyl carbonates), such as di(2-ethylhexyl) carbonate and dicaprylyl carbonate. Further suitable esters are dicarboxylic esters, such as di-n-butyl adipate, di-n-butyl sebacate, di(2-ethylhexyl) adipate, di(2-hexyldecyl) succinate, diisotridecyl acelate. Suitable diol esters are, for example, ethylene glycol dioleate, ethylene glycol diisotridecanoate, propylene glycol di(2-ethyl hexanoate), butane-diol diisostearate and neopentyl glycol dicaprylate.

Also suitable as oil component are the fatty acid triglycerides, where, among these, the naturally occurring oils and fats are preferred. Suitable oil components are, for example, natural, vegetable oils, for example, olive oil, sunflower oil, soybean oil, groundnut oil, rapeseed oil, almond oil, palm oil or else the liquid fractions of coconut oil or of palm kernel oil, and animal oils, such as, for example, neat's foot oil, the liquid fractions of beef tallow or also synthetic triglycerides of caprylic/capric acid mixtures, triglycerides of technical-grade oleic acid or of palmitic acid/oleic acid mixtures.

Suitable further auxiliaries and additives are, inter alia, UV light protection filters.

UV light protection filters are understood as meaning organic substances which are able to absorb ultraviolet rays and re-emit the absorbed energy in the form of long-wave radiation, for example, heat. UVB filters may be oil-soluble or water-soluble. Examples of oil-soluble substances are:

3-benzylidenecamphor and derivatives thereof, for example, 3-(4-methyl-benzylidene)camphor

4-aminobenzoic acid derivatives, preferably 2-ethylhexyl 4-(dimethylamino)benzoate, 2-ethylhexyl 4-(dimethylamino)benzoate and amyl 4-(dimethylamino)benzoate

esters of cinnamic acid, preferably 2-ethylhexyl 4-methoxycinnamate, isopentyl 4-methoxycinnamate, 2-ethylhexyl 2-cyano-3-phenylcinnamate (octocrylene)

esters of salicylic acid, preferably 2-ethylhexyl salicylate, 4-isopropylbenzyl salicylate, homomenthyl salicylate

derivatives of benzophenone, preferably 2-hydroxy-4-methoxybenzophenone, 2-hydroxy-4-methoxy-4′-methylbenzophenone, 2,2′-dihydroxy-4-methoxybenzophenone

esters of benzalmalonic acid, preferably di-2-ethylhexyl 4-methoxybenzalmalonate

triazine derivatives, such as, for example, 2,4,6-trianilino(p-carbo-2′-ethyl-1′-hexyloxy)-1,3,5-triazine and octyltriazone

propane-1,3-diones, such as, for example, 1-(4-tert-butylphenyl)-3-(4′-methoxyphenyl)propane-1,3-dione.

Suitable water-soluble substances are:

2-phenylbenzimidazole-5-sulfonic acid and the alkali metal, alkaline earth metal, ammonium, alkylammonium, alkanolammonium and glucammonium salts thereof

sulfonic acid derivatives of benzophenone, preferably 2-hydroxy-4-methoxybenzophenone-5-sulfonic acid and its salts

sulfonic acid derivatives of 3-benzylidenecamphor, such as, for example, 4-(2-oxo-3-bornylidenemethyl)benzene-sulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tert-butylphenyl) -3-(4′-methoxyphenyl)propane-1,3-dione or 1-phenyl-3-(4′-isopropylphenyl)propane-1,3-dione. The UV-A and UV-B filters can, of course, also be used in mixtures. In addition to the foregoing soluble substances, insoluble pigments, namely finely disperse metal oxides or salts, are also suitable for this purpose, such as, for example, titanium dioxide, zinc oxide, iron oxide, aluminum oxide, cerium oxide, zirconium oxide, silicates (talc), barium sulfate and zinc stearate. Here, the particles should have an average diameter of less than 100 nm, preferably between 5 and 50 nm and, in particular, between 15 and 30 nm. The particles may have a spherical shape, although it is also possible to use particles which have an ellipsoidal shape or a shape which deviates in some other way from the spherical form. A relatively new class of light protection filters are micronized organic pigments, such as, for example, 2,2′-methylenebis {6-(2H-benzotriazol-2-yl)-4-(1,1,3,3-tetramethylbutyl)phenol} having a particle size of <200 nm, which is available, for example, as a 50% strength aqueous dispersion.

In addition to the two above mentioned groups of primary light protection substances, it is also possible to use secondary light protection agents of the antioxidant type, which interrupt the photochemical reaction chain that is triggered when UV radiation penetrates into the skin. Typical examples thereof are amino acids (for example, glycine, histidine, tyrosine, tryptophan) and derivatives thereof, imidazoles (for example, urocanic acid) and derivatives thereof, peptides such as D,L-camosine, D-camosine and derivatives thereof (for example, anserine), carotenoids, carotenes (for example,

α-carotene, β-carotene, lycopene) and derivatives thereof, chlorogenic acid and derivatives thereof, lipoic acid and derivatives thereof (for example, dihydrolipoic acid), aurothioglucose, propylthiouracil and other thiols (for example, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiopropionate, distearyl thiopropionate, thiodipropionic acid and derivatives thereof (esters, ethers, peptides, lipids, nucleotides, nucleosides and salts) and sulfoximine compounds (for example, buthionine sulfoximines, homocysteine sulfoximine, buthionine sulfones, penta-, hexa-, heptathionine sulfoximine) in very low tolerated doses (for example, μmol to μmol/kg), and also (metal) chelating agents (for example, α-hydroxy fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA and derivatives thereof, ubiquinone and ubiquinol and derivatives thereof, vitamin C and derivatives (e.g., ascorbyl palmitate, Mg ascorbyl phosphate, ascorbyl acetate), tocopherols and derivatives (for example, vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnrosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (for example, ZnO, ZnSO₄), selenium and derivatives, thereof (for example, selenomethionine), stilbenes and derivatives thereof (for example, stilbene oxide, trans-stilbene oxide) and the derivatives (salts, esters, ethers, sugars, nucleotides, peptides and lipids) of said active ingredients which are suitable according to the present invention.

Suitable preservatives are, for example, phenoxyethanol, formaldehyde solution, parabens, pentanediol or sorbic acid.

Suitable insect repellents are N,N-diethyl-m-toluamide, 1,2-pentanediol or Insect Repellent 3535. Suitable self-tanning agents are dihydroxyacetone, and perfume oils which may be mixtures of natural and synthetic fragrances. Natural fragrances are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemons, oranges), roots (mace, angelica, celery, cardamom, costus, iris, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are e.g., benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. However, preference is given to using mixtures of different fragrances which together produce a pleasing scent. Ethereal oils of relatively low volatility, which are mostly used as aroma components, are also suitable as perfume oils, for example, sage oil, camomile oil, oil of cloves, balm oil, mint oil, cinnamon leaf oil, lime blossom oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamenaldehyde, linalool, boisambrene forte, ambroxan, indole, Hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, Evernyl, iraldein gamma, phenylacetic acid, geranyl acetate, benzyl acetate, rose oxide, Romillat, Irotyl and Floramat alone or in mixtures.

Dyes which may be used in the present invention are the substances permitted and suitable for cosmetic purposes, as listed, for example, in the publication “Kosmetische Färbemittel” [Cosmetic Colorants] from the Farbstoffkommission der Deutschen Forschungsgemein-schaft [Dyes Commission of the German Research Society], Verlag Chemie, Weinheim, 1984, pp. 81-106. These dyes are customarily used in concentrations of from 0.001 to 0.1% by weight, based on the total mixture.

Suitable deodorant active ingredients are, e.g., odor-masking agents, such as, the customary perfume constituents, odor absorbers, for example, the phyllosilicates described in laid-open specification DE-A-40 09 347, and of these, in particular, montmorillonite, kaolinite, illite, beidellite, nontronite, saponite, hectorite, bentonite, smectite, and also, for example, zinc salts of ricinoleic acid. Antibacterial agents are also suitable for incorporation into the oil-in-water emulsions according to the present invention. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichloro-carbanilide, quaternary ammonium compounds, oil of cloves, mint oil, thyme oil, triethyl citrate, farnesol (3,7,11-trimethyl-2,6,10-dodecatrien-1-ol) and the active agents described in the laid-open specifications DE-A-198 55 934, DE-A-37 40 186, DE-A-39 38 140, DE-A-42 04 321, DE-A-42 29 707, DE-A-42 29 737, DE-A-42 38 081, DE-A-43 09 372 and DE-A-43 24 219. Further customary antiperspirant active ingredients can likewise be advantageously used in the preparations according to the present invention, in particular, astringents, for example, basic aluminum chlorides, such as, aluminum chlorohydrate (“ACH”) and aluminum zirconium glycine salts (“ZAG”).

Examples of suitable active ingredients which may also be employed in the present invention are tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

The following examples are provided to illustrate the method used in forming the invention hydrophobically modified copolymeric polyglutamic acid derivatives as well as their use in forming oil-in-water emulsions.

### WORKING EXAMPLES

In a first stage, glutamic acid is melted, for example, at 170° to 190° C., during which the cyclic amide forms the pyroglutamic acid by eliminating water. Aspartic acid and/or other coamino acids are then added, and the melt is heated further at the same temperature. The water of reaction which forms is distilled off continuously. The longer the heating time and the higher the temperature, the greater the molecular mass of the resulting peptide. This peptide can also be prepared in a stepless process, where coamino acid, glutamic acid and optionally further amino acids are heated and polymerized simultaneously at the same temperature.

In the next step, amines are added to the peptide and reacted further at 170° to 190° C. The resulting melt is either poured out and comminuted after cooling, or treated directly afterward with aqueous bases, e.g., aqueous sodium hydroxide solution. The suspension obtained in this way can either be further used directly or isolated.

In one process variant, after the glutamic acid, the amine is added and only then in the following step are the aspartic acid or/and other coamino acids added.

In a further process variant, all of the components can be mixed together and reacted under the given conditions.

### Preparation Examples 1 to 8

Examples of hydrophobically modified polyglutamic acid derivatives used according to the present invention are listed below:

### Example 1

221 g of glutamic acid and 200 g of aspartic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed. 54 g of water were then distilled off. 105 g of stearylamine (Armeen 18 D from Akzo Nobel) were then slowly added over the course of half an hour, and the mixture was stirred for a further half an hour at the same temperature. At 140° C., a 25% strength sodium hydroxide solution was added until a pH of 5 had been established. The product was bleached by adding 1 g of amino-iminoethanesulfinic acid at 80° C. over the course of one hour. 10 g of 30% hydrogen peroxide solution was then added and the mixture was stirred for a further hour at 80° C.

The product was concentrated on a rotary evaporator. The residual amounts of the amino acids were determined by means of HPLC. This gave a pale beige 50% strength suspension with about 5% free aspartic acid and 18% pyroglutamic acid.

### Example 2

80 g of glutamic acid, 320 g of aspartic acid and 100 g of stearylamine (Armeen 18 D from Akzo Nobel) were introduced, under a gentle stream of nitrogen, into a 2 l flat-flange flask fitted with an oil bath, stirrer, thermometer and distillation bridge, and heated to 190° C. After 1.5 h, the temperature was increased to 220° C. and the mixture was stirred for a further 2.5 h. The water of reaction which forms (53 g) was distilled off over the distillation bridge and condensed. At 140° C., a 25% strength sodium hydroxide solution and water were added until a pH of 7 had been established. The product was bleached by adding 1 g of aminoiminomethanesulfinic acid at 80° C. over the course of one hour. 10 g of 30%-strength hydrogen peroxide solution were then added and the mixture was stirred for a further hour at 80° C. The product was evaporated on a rotary evaporator. The residual amounts of the amino acids were determined by means of HPLC. This gave a pale beige 29% strength suspension with about 14% free aspartic acid, 2% glutamic acid and 3% pyroglutamic acid.

### Example 3

250 g of glutamic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed (30 g). After 1.5 h, 119 g of stearylamine were added and the mixture was stirred at 180° C. for 0.5 h. 226 g of aspartic acid were then added and, after 1 h, 26 g of water were distilled off. At 150° C., a 25% strength sodium hydroxide solution was added until a pH of 5 had been established. Concentration on a rotary evaporator gave a pale yellow powder. The residual amounts of the amino acids were determined by means of HPLC. This gave a modified peptide with 21% free aspartic acid, 18% pyroglutamic acid and 4% free glutamic acid.

### Example 4

500 g of glutamic acid and 45 g of aspartic acid were introduced, under a gentle stream of nitrogen, into a 2 l flat-flange flask fitted with an oil bath, stirrer, thermometer and distillation bridge, and heated to 220° C. The water of reaction which forms was distilled off over the distillation bridge and condensed (67 g). 105 g of stearylamine (Armeen 18 D from Akzo Nobel) were then added slowly over the course of half an hour. At 140° C., a 25% strength sodium hydroxide solution was added until a pH of 7 had been established.

The product was concentrated on a rotary evaporator. The residual amounts of the amino acids were determined by means of HPLC. This gave a yellow powder with about 2% free glutamic acid and 50% pyroglutamic acid.

### Example 5

74 g of glutamic acid and 67 g of aspartic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed (18 g). 270 g of stearylamine (Armeen 18 D from Akzo Nobel) were then added slowly over the course of half an hour, and the mixture was stirred for a further 0.5 hours at 150° C. At 140° C., a 25% strength sodium hydroxide solution was added until a pH of 7 had been established. The product was concentrated on a rotary evaporator. The residual amounts of the amino acids were determined by means of HPLC. This gave a modified peptide with about 2% free aspartic acid and 5% pyroglutamic acid.

### Example 6

221 g of glutamic acid and 200 g of aspartic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed.

After 54 g of water had been distilled off, 307 g of dimethylaminopropylamine were slowly added over the course of one hour, and the mixture was stirred for a further one hour at the same temperature. This gave a brown solution which, after cooling, was of high viscosity and tacky.

### Example 7

200 g of glutamic acid and 200 g of aspartic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed.

After 52 g of water had been distilled off, 100 g of stearylamine (Armeen 18 D from Akzo Nobel) were slowly added over the course of half an hour, and the mixture was stirred for a further half an hour at the same temperature. 100 g of glycine were then added over the course of half an hour and again the mixture was stirred for half an hour at constant temperature. At 140° C., a 25% strength sodium hydroxide solution was added until a pH of 7 had been established. This gave a brown suspension with about 54% solids content.

### Example 8

200 g of glutamic acid and 181 g of aspartic acid were introduced, under a gentle stream of nitrogen, into a 2 l flat-flange flask fitted with an oil bath, stirrer, thermometer and distillation bridge, and heated to 180° C. The water of reaction which forms was distilled off over the distillation bridge and condensed.

After 48 g of water had been distilled off, the mixture was stirred for half an hour at the same temperature. 95 g of polyoxyalkyleneamine (Jeffamine M-600 from Huntsman) were then slowly added over the course of half an hour, and the mixture was stirred for a further half an hour at the same temperature. At 140° C., a 25% strength sodium hydroxide solution was added until a pH of 5 had been established. The product was bleached by adding 1 g of aminoiminomethanesulfinic acid at 80° C. over the course of one hour. 10 g of 30% strength hydrogen peroxide solution were then added, and the mixture was stirred for a further hour at 80° C.

The product was concentrated on a rotary evaporator. The residual amounts of the amino acids were determined by means of HPLC. This gave a 53% strength clear pale brown solution with about 5% free aspartic acid and 22% pyroglutamic acid.

Examples of oil-in-water emulsions according to the present invention are listed below in Tables 1-3:

TABLE 1

| | | |
|---|---|---|
| A | Polyglutamic acid derivative | 0.5% |
| | Preparation Example 1 | |
| | TEGIN M (Glyceryl Stearate) | 3.0% |
| | TEGO Alkanol 1618 (Cetearyl Alcohol) | 2.0% |
| | Caprylic/Capric Triglyceride | 9.0% |
| | Ethylhexyl Stearate | 4.0% |
| | Mineral oil (30 mPas) | 5.0% |
| | Tocopheryl Acetate | 1.0% |
| B | Glycerol | 2.0% |
| | Panthenol | 1.0% |
| | Allantoin | 0.1% |
| | Water | 70.2% |
| C | TEGO Carbomer 134 (Carbomer) | 0.15% |
| | Ethylhexyl Stearate | 1.6% |
| D | Sodium hydroxide (10% in water) | 0.45% |
| | Preservative, Perfume | q. s. |

TABLE 2

| | | |
|---|---|---|
| A | Polyglutamic acid derivative | 1.0% |
| | Preparation Example 6 | |
| | TEGIN M (Glyceryl Stearate) | 4.0% |
| | TEGO Alkanol 1618 (Cetearyl Alcohol) | 2.0% |
| | Caprylic/Capric Triglyceride | 9.0% |
| | Ethylhexyl Stearate | 4.0% |
| | Mineral oil (30 mPas) | 5.0% |
| B | Glycerol | 3.0% |
| | Water | 70.8% |
| C | TEGO Carbomer 141 (Carbomer) | 0.15% |
| | Ethylhexyl Stearate | 0.6% |
| D | Sodium hydroxide (10% in water) | 0.45% |
| | Preservative, Perfume | q. s. |

TABLE 3

| | | |
|---|---|---|
| A | Polyglutamic acid derivative | 1.0% |
| | Preparation Example 8 | |
| | PEG-100 Stearate | 1.0% |
| | Stearyl Alcohol | 2.0% |
| | Stearic acid | 2.0% |
| | Caprylic/Capric Triglyceride | 7.0% |
| | Ethylhexyl Stearate | 6.2% |
| | Tocopheryl Acetate | 0.3% |
| B | Glycerol | 2.0% |
| | Panthenol | 0.5% |
| | Allantoin | 0.2% |
| | Water | 77.05% |
| C | TEGO Carbomer 134 (Carbomer) | 0.1% |
| | Mineral oil (30 mPas) | 0.4% |
| D | Sodium hydroxide (10% in water) | 0.25% |
| | Preservative, Perfume | q. s. |

The examples described here of oil-in-water emulsions according to the present invention are all finely disperse emulsions with a brilliant appearance and a pleasant feel on the skin, whose pH is about 5.5 and thus corresponds to the pH of the natural acid protective mantle of the skin. The oil-in-water emulsions are further characterized by very good long-term and also low-temperature and high-temperature stability.

While the present invention has been particulary shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope and spirit of the present invention.

What we claim as new is:

1. A cosmetic or pharmaceutical oil-in-water emulsion which comprises one or more hydrophobically modified copolymeric polyglutamic acid derivatives and further auxiliaries and additives, wherein the copolymeric polyglutamic acid derivatives are prepared by simultaneous or stepwise reaction of glutamic acid and at least one further α-amino acid, α-amino acid derivative, or a mixture thereof and amines in the absence of a solvent and a catalyst.

2. A cosmetic or pharmaceutical oil-in-water emulsion, which comprises
(a) one or more hydrophobically modified copolymeric polyglutamic acid derivatives prepared as in claim 1;
(b) one or more polar waxes selected from the group consisting of fatty alcohols having 12 to 22 carbon atoms, fatty acids having 12 to 22 carbon atoms, glycerol or polyglycerol partial esters of fatty acids having 12 to 22 carbon atoms, and combinations thereof, and
(c) one or more cosmetic oils.

3. The cosmetic or pharmaceutical oil-in-water emulsion as claimed in claim 2, wherein the proportion of component (a) is between 0.1 and 2.0%, the proportion of component (b) is 0.5 to 8.0%, and the proportion of component (c) is 1.0 to 60% of the overall emulsion.

4. The cosmetic or pharmaceutical oil-in-water emulsion as claimed in claim 2, which additionally comprises stabilizers selected from the group consisting of synthetic or natural hydrocolloids.

5. The cosmetic or pharmaceutical oil-in-water emulsion as claimed in claim 2, wherein the emulsion further comprises auxiliaries and additives that are customary for cosmetic emulsions.

6. The cosmetic or pharmaceutical oil-in-water emulsion as claimed in claim 5, wherein the auxiliaries and additves are selected from the group consisting of emulsifiers, UV light protection filters, antiperspirants, deodorants, antioxidants, preservatives, insect repellents, self-tanning agents, perfume oils, dyes and active ingredients.

7. The cosmetic or pharmaceutical oil-in-water emulsion as claimed in claim 1, wherein said emulsion has a pH of about 5.5.

8. The cosmetic or pharmacetucal oil-in-water emulsion as claimed in claim 1, wherein said emulsion is biodegrable.

9. The cosmetic or pharmaceutical oil-in-water emulsion as claimed in claim 1, wherein said emulsion is finely dispersed, has a brilliant appearance and has a pleasnt feel on skin
